(19) United States
(12) Patent Application Publication
(10) Pub. No.: US 2013/0136772 A1
(43) Pub. Date: May 30, 2013

(54) REGULATION OF IMMUNE RESPONSE BY COLOCYNTHIN AND/OR ITS DERIVATIVES

(71) Applicants: Muhammed Majeed, Edison, NJ (US); Kalyanam Nagabhushanam, East Windsor, NJ (US); Anjali Pardey, Bangalore (IN); Shivakumar Ranganath, Bangalore (IN)

(72) Inventors: Muhammed Majeed, Edison, NJ (US); Kalyanam Nagabhushanam, East Windsor, NJ (US); Anjali Pardey, Bangalore (IN); Shivakumar Ranganath, Bangalore (IN)

(21) Appl. No.: 13/814,027

(22) PCT Filed: Aug. 30, 2011
(86) PCT No.: PCT/US11/49696

§ 371 Date: Feb. 4, 2013

Related U.S. Application Data
(60) Provisional application No. 61/378,450, filed on Aug. 31, 2010.

(51) Int. Cl. A61K 31/704

(52) U.S. Cl. CPC A61K 31/704
USPC 424/278.1; 514/26; 435/375

(57) ABSTRACT

The present invention relates to a method of inhibiting pro-inflammatory and T-cell mediated cytokines, said method comprising step of administering to a subject in need thereof a therapeutically effective amount of colocynthin and/or its derivatives. The invention also relates to a method of inhibiting T-cell surface markers and to a method of regulating immune response by colocynthin and/or its derivatives.

This is a 371 national phase application of PCT/US11/049696 filed 30 Aug. 2011, claiming priority to U.S. provisional application No. 61/378,450 filed 31 Aug. 2010, the contents of which are incorporated herein by reference.

### FIELD OF THE INVENTION

The present invention relates to a method of inhibiting pro-inflammatory and T-cell mediated cytokines, T-cell surface markers and regulation of immune response by colocynthin and/or its derivatives.

### BACKGROUND AND PRIOR ART

Precise regulation of immune system is important for survival of the being. Responses by T-cells, B-cells, monocytes/macrophages, neutrophils etc and production of cytokines is critical to orchestrate immune and metabolic responses during development, tissue regeneration, healing, trauma or infection and to protect our bodies against haemorrhage, ischemia, cancer and sepsis. A controlled production of pro-inflammatory cytokines, such as tumour necrosis factor-alpha (TNF-α) that trigger inflammatory responses promote local coagulation to confine infection and tissue damage (Ulloa and Tracey, 2005). However, the unrestricted production of these cytokines or the responses by the cells of immune system is more dangerous than the original injury and it is one of the principal causes of human morbidity and mortality.

In addition to cytokines, other mediators like histamine, prostaglandins, leukotrienes, bradykinin etc also play a role in triggering immune response. Thus, these serve as markers and are useful in diagnosing disease conditions, especially in those conditions where they are present at elevated levels. It is therefore essential to regulate the markers and cell responses for precise control of immune system.

Many bioactive compounds or molecules are being screened for their ability to modulate inflammation and immune functions. Although plants are a rich source of bioactive molecules, more than 90% are yet to be screened for biological activity. Conventional Western medicine relies heavily on phytomedicines: 50-60% of pharmaceutical commodities contains natural products or is synthesized from them, and more than 10% of prescription drugs contain one or more natural bioactive compounds. The wide spectrum of choices for development of new plant related products for the pharmaceutical/medicinal industry leads immediately to two questions: (a) which plants and phytomolecules from among the large number of potentially valuable bioactive compounds are realistic candidates, and (b) how to achieve value addition by way of R & D directed at commercialization.

Natural products have long been a thriving source for the discovery of new drugs due to their chemical diversity and ability to act on various biological targets. History of medicine dates back practically to the existence of human civilization. The current accepted modern medicine has gradually developed over the years by scientific and observational efforts.

The present invention relates to the use of the compound hereinafter referred as AD-B-63B isolated from Citrullus colocynthes for down-regulation/inhibition of pro-inflammatory (innate immune) and T-cell mediated (adaptive immune) cytokines. The compound is also useful in modulating immune responses and thereby is potential in the management of various diseases/disorders.

### SUMMARY OF THE INVENTION

The present invention relates to a method of inhibiting pro-inflammatory and T-cell mediated cytokines, said method comprising step of administering to a subject in need thereof a therapeutically effective amount of colocynthin and/or its derivatives. The pro-inflammatory cytokines are TNF-α and IL-1β and the T-cell mediated cytokines are IL-2, IFN-γ and IL-4.

The invention also relates to a method of inhibiting T-cell surface markers such as CD4 and CD8 as well as to a method of regulating immune response by the use of colocynthin and/or its derivatives. The immune response is innate immune response and/or adaptive immune response. Innate immune response is inhibited by reduced expression of TNF-α and IL-1β and adaptive immune response comprising T-cell and B-cell mediated responses causing depletion of leucocyte migration and phagocytosis.

### BRIEF DESCRIPTION OF ACCOMPANYING FIGURES

FIG. 1: Histograms showing percentage expression of TNF-α expression by AD-B-63B in LPS activated murine neutrophils (Histogram plot showing one representative value).

FIG. 2: Effect of multiple dose of AD-B-63B on extracellular in vivo TNF-α and IL-1β estimation in serum from treated balb/c mice. Values are expressed as means±S.E.; * p<0.01; **p<0.001 Student's ‘V test; Prednisolone (standard)-5 mg/kg.

FIG. 3: Effect of multiple dose of AD-B-63B on extracellular in vivo NO estimation in serum from treated balb/c mice. Values are expressed as means±S.E; * p<0.01; **p<0.001 Student's ‘t’ test; Prednisolone (standard)-5 mg/kg.

FIG. 4: Effect of AD-B-63B on neutrophil count in carrageenan induced pleurisy.

FIG. 5: Effect of AD-B-63B on CD4+ and CD8+ T Cell population in SRBC immunised Swiss albino mice. Number of animals in each group 6. Data is represented as 0mean±S.E. *: P<0.01, **: P<0.001.

FIG. 6: Flowcytometric data presentation of the effect of AD-B-63B on CD4+ and CD8+ T Cell population in SRBC immunised Swiss albino mice.

FIG. 7: Effect of AD-B-63B on expression of IL-2 in SRBC immunised Swiss albino mice. Number of animals in each group 6; Data is represented as mean±S.E; *: P<0.01, **: P<0.001.

FIG. 8: Flowcytometric data presentation of the effect of AD-B-63B on expression of IL-2 in SRBC immunised Swiss albino mice.

FIG. 9: Effect of AD-B-63B on IFN-gamma (Th1 cytokine) and IL-4 (Th2 cytokine) expression in SRBC immunised Swiss albino mice. Number of animals in each group 6; Data is represented as mean±S.E ; *: P<0.01, **: P<0.001.

### DETAILED DESCRIPTION OF THE INVENTION

The present invention relates to a method of inhibiting pro-inflammatory and T-cell mediated cytokines, said method comprising step of administering to a subject in need thereof a therapeutically effective amount of colocynthin and/or its derivatives.

In another embodiment of the present invention, the pro-inflammatory cytokines are TNF-α and IL-1β.

In yet another embodiment of the present invention, the T-cell mediated cytokines are IL-2, IFN-γ and IL-4.

The present invention also relates to a method of inhibiting T-cell surface markers, said method comprising step of contacting the T-cell or administering to a subject in need thereof an effective amount of colocynthin and/or its derivatives.

In another embodiment of the present invention, the T-cell surface markers are CD4 and CD8.

The present invention also relates to a method of regulating immune response, said method comprising step of administering to a subject in need thereof an effective amount of colocynthin and/or its derivatives.

In another embodiment of the present invention, the immune response is innate immune response and/or adaptive immune response.

In yet another embodiment of the present invention, the innate immune response is inhibited by reduced expression of TNF-α and IL-1β and adaptive immune response comprising T-cell and B-cell mediated responses causing depletion of leucocyte migration and phagocytosis.

The present invention also relates to a dietary supplement containing colocynthin and/or its derivatives or a composition containing colocynthin and/or its derivatives.

Immune system is governed by responses by the cells of immune system such as T-cells, B-cells, monocytes, neutrophils and mediators such as cytokines. Regulation of immune system is important to protect our body from substances which the immune system recognizes as a foreign.

Immune response may be an innate immune response or an adaptive response. In the innate response, cells provide immediate defense against infection by recognizing and responding to pathogens. In adaptive immune response, cells recognize and remember specific pathogens so that they show stronger attacks each time the pathogen is encountered. Adaptive immune system mainly involves interactions between T cells, B cells and of molecules made by them.

During infection, the immune cells migrate to the site and produce chemical factors including cytokines which are specialized chemical mediators. This activates complement cascade, removal of foreign substances by white blood cells and activation of adaptive immune response. The adaptive immune system confer long-lasting or protective immunity to the host.

Inflammation is one of the first responses of the immune system to infection. It acts as a physical barrier against infection and involves macrophages, dendritic cells, histiocytes and mastocytes. It is stimulated by chemical factors released by injured cells. Cytokines produced by macrophages and other cells of the innate immune system such as TNF-α and IL-1 mediate the inflammatory response.

Responses by T-cells, B-cells, monocytes/macrophages, neutrophils etc and production of cytokines is critical to orchestrate immune and metabolic responses during development, tissue regeneration, healing, trauma or infection and to protect our bodies against haemorrhage, ischemia, cancer and sepsis. A controlled production of pro-inflammatory cytokines, such as tumour necrosis factor-alpha (TNF-α) triggers beneficial inflammatory responses that promote local coagulation to confine infection and tissue damage (Ulloa and Tracey, 2005). However, the unrestricted production of these cytokines or responses by the cells of immune system is more dangerous than the original injury and it is one of the principal causes of human morbidity and mortality. Thus, it is important to regulate the immune responses by the cells and the mediators produced by them.

The present invention provides a method of regulating immune response by use of colocynthin and/or its derivatives. The compound isolated from Citrullus colocynthis also inhibits pro-inflammatory and T-cell mediated cytokines. The compound as such or a composition containing the compound may be used in the invention.

Citrullus colocynthis (L.) Schard (Cucurbitaceae) is an Iranian Medicinal plant that has traditionally been used for treating diabetes and other ailments well documented in the traditional system of medicine.

Citrullus colocynthis is commonly known as ‘bitter guard’, ‘colocynth ‘vine of Sodom, tumba or ‘wild gourd’ is a tropical plant that grows abundantly in the south of Iran and widely in other parts of the world. It occurs throughout India and is seen growing wild in the warm, arid and sandy tracts of north-west, central and south India and on the sea shores of the Coromandal coast, Gujrat and other parts of the western India. Members of this family are generally dioecious herbs which may be prostate or climbing stem, bearing smooth spherical fruits which are mottled green when young and somewhat yellow when ripe.

Medicinally fruits, seeds and roots are used. There are several reports on the isolation of triterpenoids, flavonoids and aliphatic compounds from the fruits, roots and seeds. Its medicinal properties are supported by several studies revealing its anticancer and anti-diabetic effects.

Several biologically active compounds are isolated from different parts of the plant such as Cucurbitacins and their glycosides from fruits and seeds.

Cucurbitacins are a group of highly structurally diverse triterpenes with a rich variety of side chain derivatives and different ring substitution patterns. They are renowned for their bitter taste but also possess a broad range of biological activities.

Colocynthin extracted from the fruits of Citrullus colocynthis is characterized as 2-O-β-D-glucopyranosyl cucurbitacin E which has the synonyms as α-elaterin-2-D-glucopyranoside or colocynthin.

In the present invention, colocynthin is shown to modulate immune responses and inhibit pro-inflammatory cytokines and T-cell mediated cytokines. The pro-inflammatory cytokines such as TNF-α, IL-1β etc are inhibited both in the invivo and invitro system by colocynthin. Inhibition of pro-inflammatory and T-cell mediated cytokines and regulation of immune response suggests the role of the compound in treating disorders/diseases requiring inhibition of cytokines and immune responses.

Derivatives of the compound may be isolated or synthesized to show the same or enhanced activity as that of the compound.

The invention is further elaborated with the help of following examples. However, these examples should not be construed to limit the scope of the invention.

### EXAMPLES

### Example 1

### Intracellular TNF-α Estimation in Murine Neutrophils by Flowcytometry

Flowcytometric studies were carried out to determine the anti-inflammatory effect of AD-B-63B on TNF-α cytokine expression in LPS activated murine neutrophils. Neutrophils play essential roles in host defence through their ability to clear bacterial infections. Their inappropriate activation contributes to a variety of inflammatory diseases, from the acute respiratory distress syndrome to asthma, chronic obstructive pulmonary disease and rheumatoid arthritis. TNF-α was chosen as the target for anti-inflammatory activity because of the fact that it is a pro-inflammatory cytokine and plays a major role in the pathogenesis of septic shock induced by LPS (Lipopolysaccharide) endotoxin injection [Remick, D. G.; Strieter, R. M.; Eskandari, M. K.; Nguyen, D. T.; Genord, M. A.; Raiford, C. L. Am. J. Path. 1990, 136, 49.]. LPS is an important triggering factor for in vivo systemic inflammatory response [Bone, R. C. Chest 1991, 100, 802]. LPS activates neutrophils via engagement of TLR4 (Toll-like receptor 4),[Sabroe, I.; Read, R. C.; Whyte, M. K. B.; Dockrell, D. H.; Vogel, S. N.; Dower, S. K. J. Immunol. 2003, 171, 1630.] resulting in the induction of a characteristic pro-inflammatory phenotype and prolongation of cell lifespan [Lee, A.; Whyte, M. K. B.; Haslett, C. J. Leukoc. Biol. 1993, 54, 283].

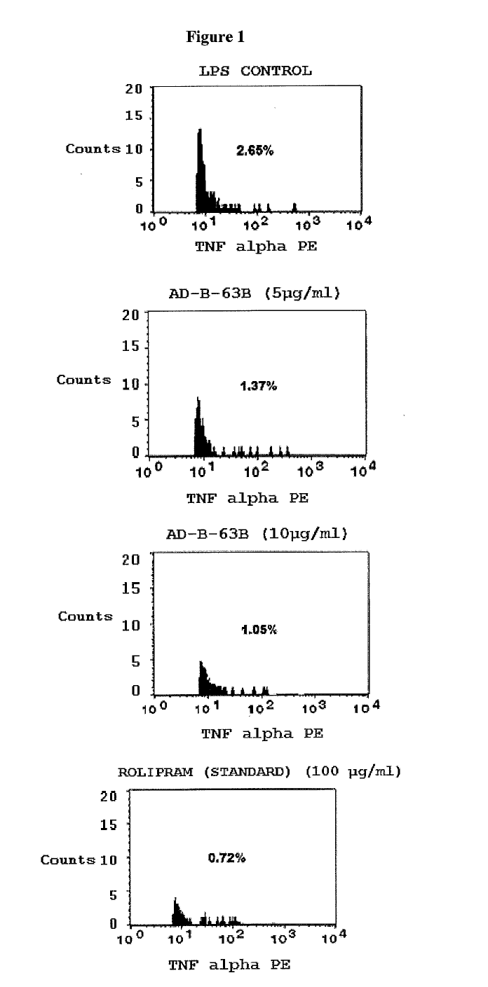

The anti-inflammatory activity of AD-B-63B was measured by the estimation of intracellular tumor necrosis factor alpha (TNF-α) expression in a gated population of neutrophils [Clara, B., R. C. Arancha, G. M. Andre's, P. Atanasio, A. Julia, and O. Alberto. 2003. A new method for detecting TNF-α-secreting cells using direct immunofluorescence surface membrane stainings. J. Immuno. Methods 264:77-87.]. Murine blood was subjected to centrifugation at 250× g for 20 min. Three layers were formed: an upper layer of platelet-rich plasma, a buffy coat middle layer and a lower layer formed of red blood cells. The middle layer was removed and subjected to Histopaque 1077 (Sigma) gradient separation. The upper layer containing neutrophils was removed and transferred to FACS tubes (Becton Dickinson). Lipopolysaccharide (LPS) derived from Escherichia coli (Sigma) was added at a concentration of 10 ng/ml for the stimulation of the cells. AD-B-63B was added at graded concentrations of 0.5, 1, 2.5, 5, 10 and 20 μg/ml. Samples were incubated for 3 h at 37° C. ( Table 1) Controls consisted of LPS-stimulated cells (LPS control). Further processing was done by the addition of FACS permeabilizing solution (Becton Dickinson), followed by the addition of phycoerythrin (PE)-labeled anti-mouse TNF-α. (Becton Dickinson). The cells were incubated in the dark, and after being washed with sterile PBS, samples were resuspended in PBS (pH 7.4) and acquired directly on the flow cytometer (FACS CANTO; Becton Dickinson). A fluorescence trigger was set on the PE (FL1) parameter of the gated neutrophil populations (10,000 events). Rolipram at 100 μg/ml was used as standard inhibitor of TNF-α in this study. Fluorescence compensation, data analysis and data presentation were performed using software (Becton Dickinson) (FIG. 1) [Pandey A, Bani S, Kaul S, Sangawan P L; 2010, Selective Th1 upregulation by ethyl acetate fraction of Labisia pumila; 132 (2010) 309-315].

TABLE 1
Effect of AD-B-63B at graded doses on expression of Intracellular Tumor
necrosis factor - alpha in LPS activated murine neutrophils.

| | | | | % TNF-alpha |
|---|---|---|---|---|
| | | | | inhibition |
| | | Concentration | | against LPS |
| S. No. | Samples | (μg/ml) | Mean ± S.E | control |
| 1 | LPS Con | — | 2.69 ± 0.06 | — |
| 2 | AD-B-63B | 0.5 | 2.34 ± 0.07 | 13.01↓ |
| 3 | AD-B-63B | 1.0 | 2.04 ± 0.06* | 24.16↓ |
| 4 | AD-B-63B | 2.5 | 1.84 ± 0.03** | 31.59↓ |
| 5 | AD-B-63B | 5.0 | 1.33 ± 0.06*** | 50.55↓ |
| 6 | AD-B-63B | 10.0 | 1.06 ± 0.03*** | 60.59↓ |
| 7 | Rolipram | 100 | 0.74 ± 0.04*** | 72.49↓ |
| | (standard) | | | |

No. of Observations-3;
LPS Con: Lipopolysaccharide Control;
AD-B-63B: 0.5, 1, 2.5, 5, 10 μg/ml;
*p < 0.05;
**p < 0.01,
***p < 0.001;
Student's ‘t’ test;
↓—Decrease in intracellular TNF alpha expression in murine neutrophils

The results showed significant decrease in intracellular TNF α expression in LPS activated neutrophils by AD-B-63B in a dose related manner with the maximum effect at the higher dose level of 5 and 10 μg/ml where the percentage inhibition was 50.55% and 60.59% respectively.

### Example 2

### Effect of AD-B-63B on Expression of Extracellular Interleukin-1Beta (IL-1β) in Murine Neutrophils (In Vitro)

IL-1beta is a major immune response modifiers produced primarily by activated microphages and macrophages. IL-1beta induces the expression of other autocrine growth factors, increases cellular responsiveness to growth factors and induces signalling pathways that lead to proliferation. All published data agree that biosynthesis of IL-lbeta increases in animal models of inflammatory diseases and inhibitors of IL-lbeta show strong prophylactic and anti-inflammatory activity. Like other growth factors, IL-1beta induces expression of several interleukins.

Neutrophils were separated from whole blood by Histopaque gradient method (Histopaque-No. 1077). Cell count was adjusted to approximately 10⁵/500 ml. Lipopolysaccharide, at the concentration of 10 ng/ml was used as stimulus in in-vitro studies. Samples were incubated with AD-B-63B at doses of 0.5, 1, 2.5, 5 and 10 μg/ml for 4 hr in CO₂ incubator at 37° C. and 5% CO₂ Supernatant was collected by spinning at 300×g for 10 mins. Extracellular IL-1β was estimated through ELISA (Table 2) [Weir, R. E., Morgan, A. R., Britton, W. J., Butlin, C. R., Dockrell, H. M., 1994. Development of a whole blood assay to measure T cell responses to leprosy: a new tool for immuno-epidemiological fields studies of leprosy immunity. J. Immunol. Methods. 176, 93; Hussain, R., Kaleem, A., Shahid, F., Dojki, M., Jamil, B., Mehmood, H., Dawood, G., Dockrell, H., 2002. Cytokine profiles using whole blood assays can discriminate between tuberculosis patients and healthy endemic controls in a BCG-vaccinated population. J. Immunol. Methods. 264, 95-108]

TABLE 2
Effect of multiple dose of AD-B-63B on expression of extracellular
Interleukin-1beta (IL-1β) in murine neutrophils

| | | | | % activity |
|---|---|---|---|---|
| | | | Concentration | IL-1β |
| | | Concentration | of IL-1β | expression |
| | | of Test sample | produced (pg/ml) | against LPS |
| S. no | Samples | (μg/ml) | Mean ± S.E. | control |
| 1 | LPS Control | — | 134.72 ± 2.39 | — |
| 2 | AD-B-63B | 0.5 | 120.91 ± 2.61 | 10.25↓ |
| 3 | AD-B-63B | 1 | 109.58 ± 3.21 | 18.66↓ |
| 4 | AD-B-63B | 2.5 | 106.66 ± 2.44* | 20.82↓ |
| 5 | AD-B-63B | 5.0 | 93.08 ± 2.61* | 30.90↓ |
| 6 | AD-B-63B | 10.0 | 87.61 ± 1.99** | 34.96↓ |
| 7 | Rolipram | 100 | 63.52 ± 2.07** | 52.85↓ |
| | (standard) | | | |

No. of Observations-3;
LPS Con: Lipopolysaccharide Control;
AD-B-63B: 0.5, 1, 2.5, 5, 10 μg/ml
*p < 0.01,
**p < 0.001;
Student's ‘t’ test ↓—Decrease in extracellular IL-1β expression in murine neutrophils

AD-B-63B significantly suppressed extracellular IL-1β expression with the maximum effect at the higher dose of 5 μg/ml and 10 μg/ml showing percent inhibition of 30.90% and 34.96% respectively.

### Example 3

### Effect of AD-B-63 B on In Vivo TNF-α, IL-1 Beta and Nitric Oxide (NO) Estimation in Serum from the Treated Mice

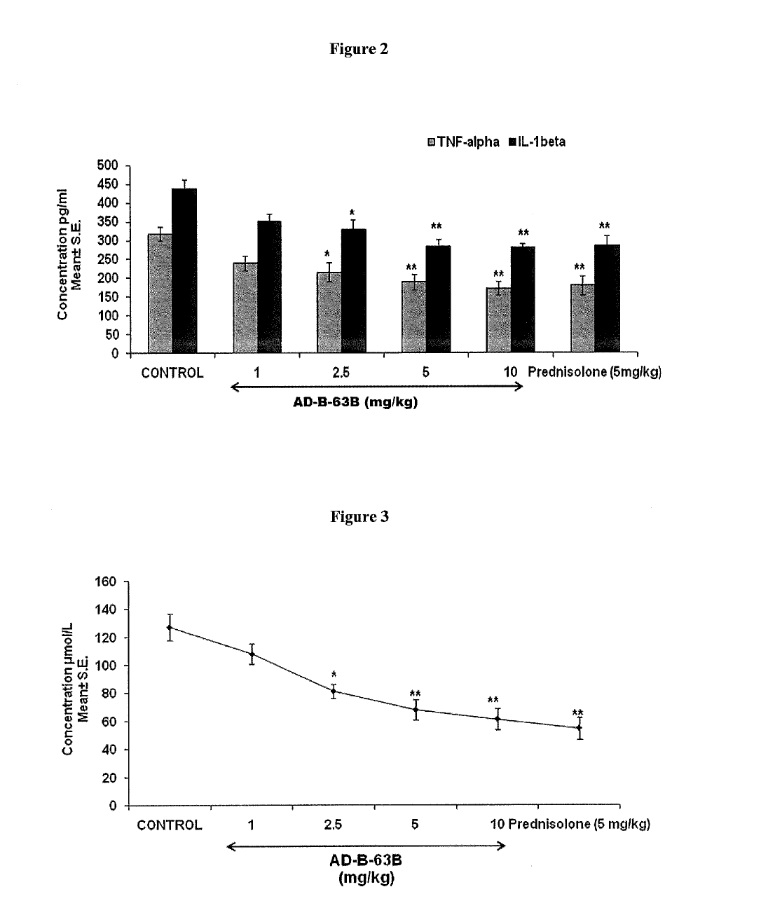

Invivo experiments were carried out using balb/c mice that were orally administrated with graded oral doses of AD-B-63B (1, 2.5, 5 and 10 mg/kg) for 6 days and challenged with LPS. Nitric oxide (NO) is an important mediator of diverse physiologic and pathologic processes, including many inflammatory diseases [Abramson S B, Amin A R, Clancy R M, Attur M, (2001). The role of nitric oxide in tissue destruction. Best Practice & Research Clinical Rheumatology; 15: 831-845]. Several lines of evidence implicate NO in the pathogenesis of inflammation. For example, rodents in animal models of arthritis generate abundant quantities of NO, as reflected in high levels of serum and urinary NO that develop in association with disease manifestations [Stefanovic-Racic M, Meyers K, Meschter C, Coffey J W, Hoffman R A, Evans C H, (1994). Monomethyl arginine, an inhibitor of nitric oxide synthase, suppresses the development of adjuvant arthritis in rats. Arthritis & Rheumatism; 37: 1062-1069.] [McCartney-Francis N, Allen J B, Mizel D E, Albina J E, Xie Q W, Nathan C F, Wahl S M, (1993). Suppression of arthritis by an inhibitor of nitric oxide synthase. Journal of Experimental Medicine; 178: 749-754.] [Weinberg J B, (1998). Nitric oxide as an inflammatory mediator in autoimmune MRL-1 pr/1 pr mice. Environmental Health Perspectives; 106(Suppl 5): 1131-1137.]. Treatment of these animals with NOS inhibitors or NO quenchers suppresses NO production and effectively abrogates joint inflammation. BALB/c male mice aged 6-8 weeks were maintained at 22±2 ° C. under 12/12 h light dark cycle. Mice received oral treatment of 1, 2.5, 5 and 10 mg/kg of AD-B-63B (w/v) for 6 days, followed by intravenous injection of 1 mg/kg of LPS according to the method described by Brieva et al., 2001 [Brieva A, Guerrero A, Alonso-Lebrero J L and Pivel J P. 2001. Inmunoferon, a glycoconjugate of natural origin, inhibits LPS-induced TNF-α production and inflammatory responses. International Immunopharmacology 1., 1979-1987.]. Six mice were employed in each group and experiments were performed in triplicates. TNF-α, IL-1 beta and Nitric oxide production was evaluated by a commercial ELISA kits (R&D Systems) in serum from AD-B-63B treated mice, 90 min after LPS injection. Prednisolone at 5 mg/kg was used as a standard drug (FIGS. 2 and 3).

Serum collection and measurements revealed a significant reduction in the levels of serum TNF-α, IL-1 beta and NO which suggests a broad, species-independent in vivo efficacy for AD-B-63B in the control of the inflammatory response. Together, these data suggest a regulatory role of the test compound in the response to increased LPS concentration in blood, not only in the TNF-α production level, but was further confirmed by reduced levels of IL-1 beta, another pro inflammatory cytokine, and NO in LPS-challenged mice.

These findings demonstrate AD-B-63B to have a potent activity in down-regulating the inflammatory markers and this is suggestive of its possible further development as moiety for therapeutic usefulness.

### Example 4

### Effect of AD-B-63B on In Vivo Leucocyte Migration

Cell migration is a cardinal feature in acute as well as chronic form of inflammatory response. In acute response, the population of white blood cells which emigrate into the inflamed tissue is mainly composed of polymorphonuclear leucocytes (PMNLS); later, the mononuclear leucocytes become dominant in the ‘inflammatory exudates. The inhibition of these leucocytes account for part of the anti-inflammatory response shown by drugs. This method has been considered to be suitable to measure chemotaxis in vivo [Blackham A, Owen R T, (1975). Prostaglandin synthetase inhibitors and leucocytic emigration. Journal of Pharmacy and Pharmacology; 27: 201-203.] The accumulation of leucocytes in the pleural cavity has been attributed to the production of chemotactic factors derived from complement following its activation by carrageenan [DiRosa M, Giroud J P, Willoughby D A, (1971). Studies on the mediators of the acute inflammatory response induced in rats in different sites by carrageenin and turpentine. Journal of Pathology; 104: 15-29.] Leucocytes then sustain the reaction by phagocytosing carrageenan and releasing the inflammatory mediators.

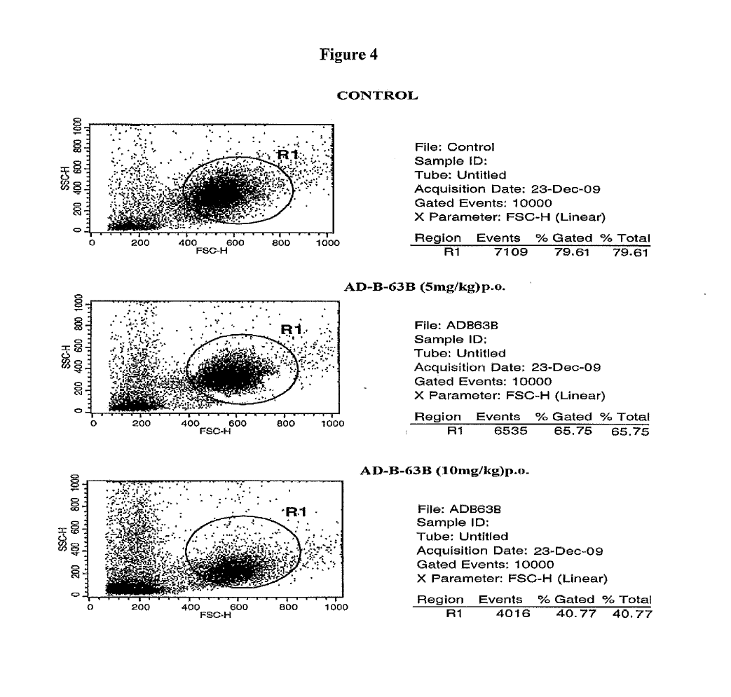

Mice were injected with 0.2 ml of carrageenan (1% w/v) solution in sterilised normal saline into the pleural cavity by the modified method of Meacock and Kitchen, 1979 [Meacock S C R, Kitchen E A, (1979). Effect of non-steroidal antiinflammatory drug benoxaprofen on leukocyte migration. Journal of Pharmacy & Pharmacology; 31: 366-370]. The test drug was administered orally 1 h before and 6 h after the injection. After 24 h of carrageenan injection the pleural exudate was collected, volume measured and the total leucocyte count of the pleural fluid was determined (Table 3). Flowcytometry as a tool for evaluation of neutrophils is particularly useful because of their larger size than monocytes and lymphocytes when analysed on the forward scatter [FSc]. The percentage of these can be analysed by selectively ‘gating’ the neutrophils without physical separation of the cells (FIG. 4).

AD-B-63B showed a dose dependent inhibition of both exudates volume and the total leucocytes count against control depending on the dose given with highly significant effect at 10 mg/kg oral dose.

### Example 5

### Effect of AD-B-63B on Vascular Permeability in Mice

Acetic acid-induced increased vascular permeability in mouse model is a capillary permeability assay. Reduction in the increased peritoneal vascular permeability indicates the suppression of the vascular response in the process of acute inflammation.

Mice were injected with 0.2% solution of Evans blue dye (0.25% w/v in normal saline) intravenously after 30 min of oral administration of the drug. Fifteen minutes later, the mice were injected intraperitoneally (1 ml/100 g of body weight) with freshly prepared 0.6% of acetic acid (v/v) in normal saline. After 30 min of acetic acid injection, the peritoneal cavities were washed with 5 ml of heparinised sterile normal saline and centrifuged (3000×g) for 10 min Absorbance of the supernatant was measured at 610 nm using a spectrophotometer (Table 3) [Whittle B. A. The use of changes in capillary permeability in mice to distinguish between narcotic and non-narcotic analgesics. Brit J Pharmacol 1964; 2: 246-253].

AD-B-63B showed a dose dependent inhibition of dye concentration against control depending on the dose given with highly significant effect at 5 and 10 mg/kg oral dose where the effect was 32.14 and 35.71% respectively. Prednisolone at 5 mg/kg p.o. showed 32.14% inhibition.

TABLE 3
Effect of AD-B-63B on in vivo leucocyte migration and acetic - acid induced
vascular permeability

| | | ^{a}TOTAL | | |
|---|---|---|---|---|
| | | LEUCOCYTE | ^{a}EXUDATE | ^{b}DYE |
| | DOSE | COUNT × 10⁻³/cmm | VOLUME(ml) | CONCENTRATION |
| TREATMENT | mg/kg p.o. | Mean ± S.E. | Mean ± S.E. | μg/25 g mouse |
| CONTROL | — | 27.46 ± 0.40 | 1.10 ± 0.10 | 0.56 ± 0.04 |
| AD-B-63-B | 1 | 24.13 ± 0.48 | 0.85 ± 0.10 | 0.50 ± 0.02 |
| | | (12.12%↓) | (22.72%↓) | (10.71%↓) |
| AD-B-63-B | 2.5 | 21.80 ± 0.60 | 0.70 ± 0.16 | 0.42 ± 0.04* |
| | | (20.61%↓) | (36.36%↓) | (25.00%↓) |
| AD-B-63-B | 5 | 21.00 ± 0.58** | 0.68 ± 0.18* | 0.38 ± 0.02** |
| | | (23.52%↓) | (38.18%↓) | (32.14%↓) |
| AD-B-63-B | 10 | 20.76 ± 0.40** | 0.61 ± 0.10** | 0.36 ± 0.02** |
| | | (24.39%↓) | (44.54%↓) | (35.71%↓) |
| Prednisolone | 5.0 | 19.00 ± 0.60** | 0.58 ± 0.28** | 0.38 ± 0.02** |
| (standard) | | (33.23%↓) | (47.27%↓) | (32.14%↓) |

% inhibition depicted in parenthesis. Number of animals in each group: 6
^{a}Drug twice administered, 1 h before carrageenan injection into the pleural cavity and 6 h after carrageenan injection in invivo leucocyte migration test in mice.
P value: *<0.01; **<0.001
^{b}Data shows increase in concentration of dye due to excessive vascular permeability (mean ± SE) in mice, and within parentheses, the percent (mean ± SE) reduction of dye concentration by drug treatment.

### Example 6

### Effect of AD-B-63B on Macrophage Phagocytic Response

Macrophages survey the body for foreign antigens, which they destroy by making toxic molecules such as the reactive oxygen intermediate molecules. Continued production of these toxic molecules by overactive macrophages not only destroys the foreign antigens but also the tissues surrounding them The reduced responsiveness of macrophages involved is evident by the decrease in clearance of carbon particles from the reticuloendothelial system and also reduction in the rate of phagocytosis in vitro by murine macrophages, thereby, suggesting the reduction in the functioning of macrophages after treatment with AD-B-63B (Table 4). CD4+ T cell inhibition by AD-B-63B may be one of the factors responsible for the decrease in the functioning of the macrophages as the activation of primary cells of phagocytosis is one major effector function of CD4+ T cells.

### In Vitro

The method of Lehrer (1981) [Lehrer, R. I. Ingestion and destruction of Candida albicans. (1981) In: Methods for studying mono-nuclear phagocytes. Eds. Adams, D. O., Edelson, P. J. and Koren, H. (Academic Press, New York) 693-7081 was followed. The percentage and average number of Candida albicans cells (heatkilled) ingested by peritoneal murine macrophages was calculated. AD-B-63B was tested at the doses of 1, 2.5, Sand 10 μg/mL (Table 4).

Significant decrease in phagocytosis was observed at the dose of 5 μg/mL and 10 μg/mL, where the effect was 32.04% and 35.35% respectively. Prednisolone at 5 μg/mL showed 40.35% decrease in phagocytosis of heat killed Candida albicans by the murine macrophages.

### In Vivo

The phagocytic clearance of the endothelial system was assayed in groups of six mice each by injecting i.v. 160 mg/kg of 1.6% suspension of gelatin stabilized carbon particles [Atal, C. K., Sharma, M. L., Kaul, A and Khajuria, A. (1986) Immunomodulating agents of plant origin. 1. Preliminary screening. Journal of Ethnopharmacology. 18, 133-141.] Blood samples were collected before and at intervals varying between 2 and 90 min after carbon injection. An aliquot (10 μL) of blood samples were lysed with 2mL of 0.1% acetic acid and the transparency determined spectrophotometrically at 675 nm (Uvikon 810, spectrophotometer, Kontron Ltd., Switzerland) (Hudson and Hay, 1980). AD-B-63B was administered orally for 7 days and 30 min prior to the carbon injection. The rate of carbon clearance termed as phagocytic index was calculated (Table 4).

Oral administration of AD-B-63B for 7 days decreased the clearance rate of carbon particles from circulation in mice. The decrease in phagocytic index was 14.28-26.19% at dose range of 1-10 mg/kg. The effect was statistically highly significant at higher doses.

TABLE 4
Effect of AD-B-63B on Phagocytic function of macrophages in mice

| Phagocytosis of heat killed Candida albicans | | | Clearance rate of carbon particles from | | |
|---|---|---|---|---|---|
| by the murine macrophages | | | the circulation in normal animals | | |
| | | % | | | Phagocytic |
| | | phagocytosis | | | index [in vivo |
| | Dose | [in vitro study] | | Dose | study] |
| Treatment^{a} | μg/ml | Mean ± S.E. | Treatment^{b} | mg/kg | Mean ± S.E. |
| CONTROL | — | 26.81 ± 2.02 | CONTROL | | 1.26 ± 0.12 |
| AD-B-63B | 1 | 24.00 ± 2.32 | AD-B-63B | 1 | 1.08 ± 0.09 |
| | | (10.48%↓) | | | (14.28%↓) |
| AD-B-63B | 2.5 | 22.50 ± 1.95 | AD-B-63B | 2.5 | 0.97 ± 0.14 |
| | | (16.07%↓) | | | (23.01%↓) |
| AD-B-63B | 5 | 18.22 ± 2.11 | AD-B-63B | 5 | 0.92 ± 0.04 |
| | | (32.04%↓) | | | (26.98%↓) |
| AD-B-63B | 10 | 17.33 ± 1.42 | AD-B-63B | 10 | 0.93 ± 0.07 |
| | | (35.35%↓) | | | (26.19%↓) |
| PREDNISOLONE | 5 | 15.99 ± 2.17 | PREDNISOLONE | 5 | 0.84 ± 0.09 |
| | | (40.35%↓) | | | (33.33%↓) |

Data is represented as mean ± S.E;
Figures in paranthesis represents percentage change;
↓percent decrease;
*P < 0.01,
**P < 0.001;
^{a}number of observations for each dose 6.
^{b}number of animals in each group 6.

### Example 7

### Effect of AD-B-63B on Skin Allograft Rejection

The modified method of Billingham and Medawar (1951) [Billingham M E J, Davies G E. Experimental models of arthritis in animals as screening tests for drugs to treat arthritis in man. Handbook Exp Pharmacol 1979; 50(2):108-44] was followed to study the skin allograft rejection time in mice. Graded doses of test material AD-B-63B were administered to the animals for 7 days and graft rejection time (GRT) was recorded by daily observation of epithelial skin layer survival. Control group was given vehicle only, and another group received prednisolone as standard at 5 mg/kg body weight daily for 7 days (Table 5). The basic mechanism involved in increase in graft rejection time is the suppression of T lymphocytes. After transplantation, MHC peptide complexes of the foreign organ are recognized by CD4+ and CD8+ T cells of the recipient as non-self antigens. These T cells differentiate into effector T cells and stimulate an immune response. After T cells differentiate and migrate to the site, macrophages and other inflammatory agents are mediated to the site. Cytotoxic T cells (CD8+) lyse the endothelial cells on the graft, CD4+ Th1 activate macrophages and CD4+ Th2 aid in antibody production. T cells expressing CD4 are increased when there is a general expansion due to active immunological activity of the T cell and inhibition of this observation shows immunosuppressive activity.

Oral administration of AD-B-63B at 1, 2.5, 5 and 10 mg/kg delayed the skin allograft rejection time in mice (days) by 16.30, 23.84, 29.23 and 31.84%, respectively. Prednisolone at 5 mg/kg increased the rejection time by 39.69%.

TABLE 5
Effect of AD-B-63B on skin allograft rejecion in mice

| | | REJECTION | |
|---|---|---|---|
| | DOSE | TIME | GRAFT |
| | mg/kg | (DAYS) | REJECTION |
| TREATMENT | p.o. | Mean ± S.E. | (Percent) |
| CONTROL | — | 13.00 ± 0.25 | — |
| AD-B-63B | 1 | 15.12 ± 0.11 | 16.30↓ |
| AD-B-63B | 2.5 | 16.10 ± 0.21 | 23.84↓ |
| AD-B-63B | 5 | 16.80 ± 0.22 | 29.23↓ |
| AD-B-63B | 10 | 17.14 ± 0.23 | 31.84↓ |
| PREDNISOLONE | 5 | 18.16 ± 0.16 | 39.69↓ |
| (STANDARD) | | | |

Data is represented as mean ± S.E;
↓Delayed;
*P < 0.01,
**P < 0.001;
Student's ‘t’ test number of animals in each group 6.

### Example 8

### Effect of AD-B-63B on Cell Mediated Immune response (Delayed Type Hypersensitivity (DTH) Response)

Cell-mediated immunity (CMI) is a T-cell-mediated defense mechanism against microbes that survive within phagocytes or infect non phagocytic cells. CMI functions to enhance actions of phagocytes to eliminate microbes. CMI manifests as delayed type cellular immune responses the activation of which depends on interferon gamma (IFN-γ), a major cytokine produced by CD4⁺ T-helper (Th1) cells. The inhibition of CMI response inhibits the actions of phagocytes like macrophages and neutrophils (innate immune response) and retards the activation of CD4+ T helper cells by inhibiting T cell mediated cytokines (adaptive immune response).

Test material was administered 2 h after immunization of mice by 200 μl of 5×10⁹ SRBCs/ml. On 7^{th} day, the thickness of the left hind foot was measured with spheromicrometer (0.01 mm pitch) and was considered as control. These mice were then challenged by injecting the 20 μl of 5×10⁹ SRBCs/ml intradermally into the left hind footpad. The footpad thickness was measured again at 24 h after challenge (Doherty, 1981). Orally administered AD-B-63B showed suppression in DTH response (Table 6).

AD-B-63B produced a dose dependent decrease in SRBC induced DTH reaction showing the immunosuppressive effect on T-lymphocytes and accessory cell types required for the expression of the reaction.

TABLE 6
Effect of AD-B-63B on SRBC induced Delayed type hypersensitivity
(DTH) response (CMI) in mice

| | | FOOT PAD | |
|---|---|---|---|
| | DOSE | THICKNESS | PERCENT |
| TREATMENT | (mg/kgp.o.) | (mm) Mean ± SE | INHIBITION |
| CONTROL | — | 0.92 ± 0.02 | — |
| AD-B-63-B | 1 | 0.78 ± 0.01 | 15.21↓ |
| AD-B-63-B | 2.5 | 0.72 ± 0.03* | 21.74↓ |
| AD-B-63-B | 5 | 0.66 ± 0.01** | 28.26↓ |
| AD-B-63-B | 10 | 0.65 ± 0.02** | 29.35↓ |
| Prednisolone | 5 | 0.59 ± 0.05** | 35.86↓ |
| (standard) | | | |

Number of animals in each group 6.
Data is represented as mean ± S.E
*P < 0.01,
**P < 0.001,
↓decrease.

### Example 9

### Effect of AD-B-63B on SRBC Induced Humoral Antibody Response

The term “humoral” refers to the non-cellular components of the blood, such as plasma and lymphatic fluid. The humoral immune response denotes immunologic responses that are mediated by antibodies. However, both B and T lymphocytes, as well as dendritic cells and other antigen presenting cells, are necessary for the formation of antigen-specific antibody and the inhibition of this response shown by the haemagglutination antibody titre shows immunosuppressive effect of the test material on adaptive immune response.

200 μl of 5×10⁹ SRBC/ml was used to immunize the animals on day 0. Animals were treated with graded doses of AD-B-63B i.e., 1, 2.5, 5 and 10mg/kg p.o. for next six consecutive days. Blood samples were collected on day 7 for antibody titre assay. Haemagglutination antibody titre was determined following the microtitration technique described by Nelson and Midenhall, 1967. The value of highest serum dilution causing visible Haemagglutination was taken as titre. BSA saline alone served as control (Table 7).

AD-B-63B produced inhibition of antibody synthesis with a significant decrease being observed at 2.5, 5 and 10 mg/kg p.o. The inhibition of the humoral response against SRBC by AD-B-63B as evidenced by the decrease in the haemagglutination antibody titre also indicate the reduced responsiveness of macrophages and subsets of T and B lymphocytes involved in antibody synthesis thereby showing immunosuppressive nature of AD-B-63B.

TABLE 7
Effect of AD-B-63B on SRBC induced Hemagglutinin Antibody titre
response (Humoral immune response) in mice

| | | ANTIBODY | |
|---|---|---|---|
| | DOSE (mg/kg) | TITRE | |
| TREATMENT | p.o | MEAN ± S.E | % ACTIVITY |
| CONTROL | — | 6.16 ± 0.16 | — |
| AD-B-63-B | 1 | 5.33 ± 0.45 | 13.47↓ |
| AD-B-63-B | 2.5 | 4.33 ± 0.77** | 29.70↓ |
| AD-B-63-B | 5 | 4.12 ± 0.33** | 33.11↓ |
| AD-B-63-B | 10 | 4.00 ± 0.31** | 35.06↓ |
| PREDNISOLONE | 5 | 3.99 ± 0.22** | 35.22↓ |
| (STANDARD) | | | |

Number of animals in each group 6.
Data is represented as mean ± S.E
*P < 0.01,
**P < 0.001,
↓decrease.

### Example 10

### Effect of AD-B-63B on Lymphocyte Immunophenotyping

Immunophenotyping focuses on lymphocyte populations involved in acquired immunity. Specific molecules present on the cell surface defines characteristics of lymphocytes such as state of activation or functional capabilities. Murine monoclonal antibodies conjugated to a fluorochrome and directed against co-receptors CD4 and CD8 were used in a multi parametric flowcytometric assay to quantify the lymphocyte subsets associated with the cell-mediated immune response.

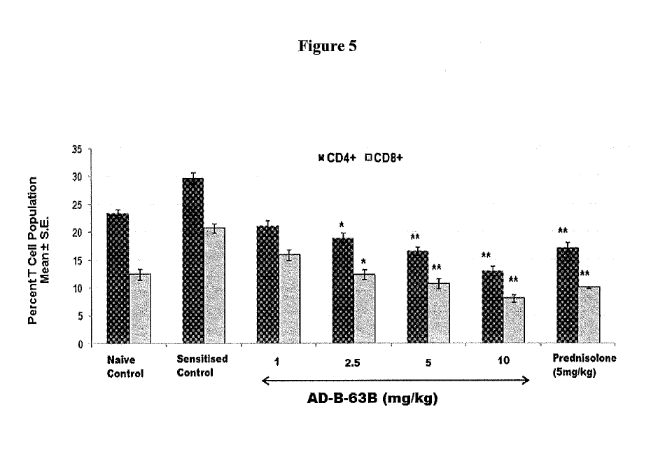

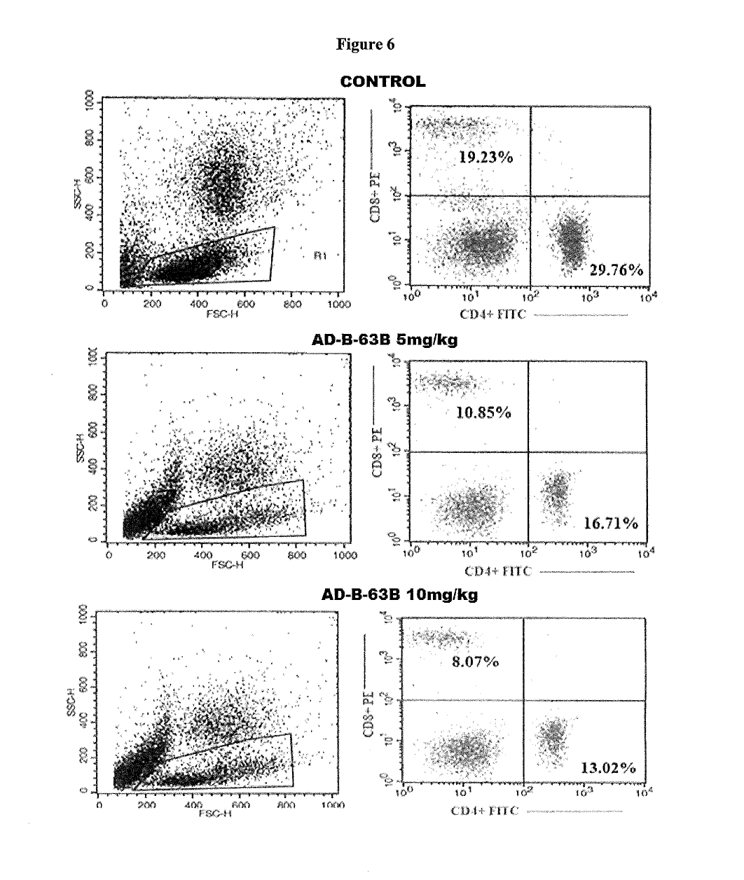

Estimation of T cell surface markers: On day 0, mice were immunized by injecting 200 μl of 5×10⁹ SRBC/ml intraperitonially. Drug administration was carried out for 6 days including the day of immunization. Same amount of SRBC was used to challenge the animals on day 7. After 24 h of challenge, blood was taken from retro-orbital plexus of animals for estimation of CD4⁺ and CD8⁺ T cells. 100 μl of whole blood was taken in each tube and FITC (fluorescein isothiocyanate) labelled CD4+ and PE (phycoerythrin) labelled CD8+ monoclonal antibodies were added and mixed gently. Tubes were incubated in the dark for 30 min. at room temperature. Subsequently, 2 ml of 1× FACS lysing solution was added at room temperature with gentle mixing. Samples were again incubated at room temperature for 10 min. After final centrifugation (300-400 g) at room temperature, the resulting stained cell pellet was resuspended in 500 μl of PBS and was run on a flow cytometer (FACS CANTO H, BD Biosciences). Acquisition and the analysis were done directly on a flow cytometer using Cell Quest Pro software [Bath S, Kaul A, Khan B, Ahmad S F, Suri K A, Satti N K, (2005). Imunosuppressive properties of an ethyl acetate fraction from Euphorbia royleana. Journal of Ethnopharmacology; 99: 185-192.] [Bani S, Kaul A, Khan B, Ahmad S F, Suri K A, Satti N K, Amina M, Qazi G N (2005). Immunosuppressive properties of an ethyl acetate fraction from Euphorbia royleana. Journal of Ethnopharmacology; 99: 185-192. ] (FIG. 5 and FIG. 6),

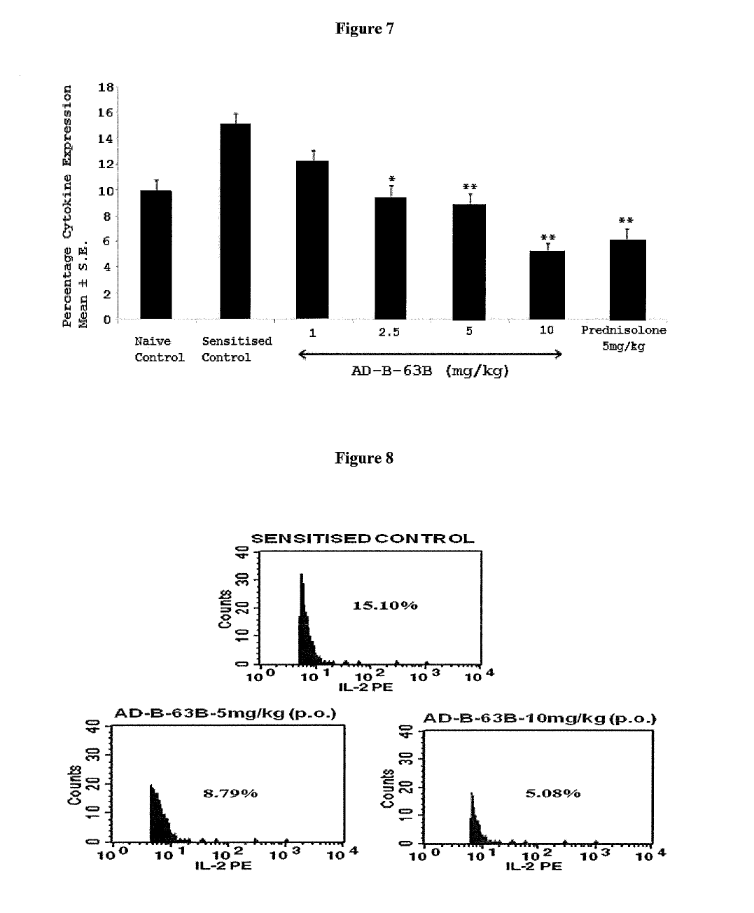

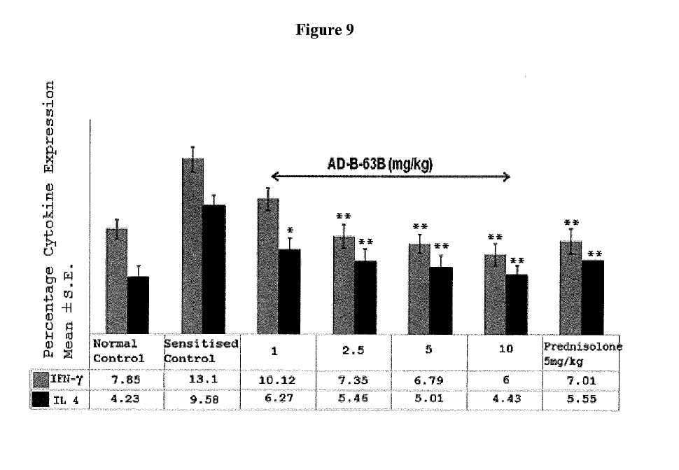

Estimation of intracellular cytokines: The animals were immunized as mentioned above. After 6 days of drug administration, the animals were challenged on day 7. After 48 h of challenge, the blood was collected in the tubes containing EDTA (anti-coagulant) from retro-orbital plexes of animals to determine the effect of test material on the expression of intracellular IL-2 and IFN-γ (Th1) and IL-4 (Th2) cytokines. In this study, PE labeled IL-2 monoclonal antibody was used in one set and PE labeled IFN-γ and FITC labelled IL-4 monoclonal antibody was used in the other set of experimentation (FIG. 7, 8, 9).

The percentage of CD4⁺ and CD8⁺ T-cell population was found to decrease in case of animals treated with graded doses of AD-B-63B. The most significant suppression was observed at the dose level of 5 and 10 mg/kg when compared with the sensitized control.

IL-2 and IFN gamma (Th1) and IL-4 (Th2) cytokines were assayed and the expression of these cytokines showed decrease in animals treated with graded doses of AD-B-63B. The most significant suppression of intracellular IL-2 was at dose of 10 mg/kg p.o. Inhibition of IFN-γ expression was observed at dose level of 5 and 10 mg/kg p.o. The expression of intracellular IL-4 was significantly inhibited at dose levels of 2.5, 5 and 10 mg/kg.

Since the inhibition of cell mediated immune response particularly CD4+ T cells and expression of intracellular IL-2 showed a strong correlation during test drug treatment, the immunosuppressive effect of AD-B-63B is due primarily to the inhibition of CD4+ T cells. Although the use of effector functions of T-cells is now one of the more promising innovative therapeutic strategies, the idea of inhibiting Th1 dominated (IL-2 and IFN-γ) and Th2 dominated (IL-4) responses by AD-B-63B suggests its possible development as a therapeutic agent in the treatment of disease conditions requiring modulation/regulation of host immune response.

1. A method of inhibiting pro-inflammatory and T-cell mediated cytokines, said method comprising step of administering to a subject in need thereof a therapeutically effective amount of colocynthin and/or its derivatives.

2. The method as claimed in claim 1, wherein the pro-inflammatory cytokines are TNF-α and IL-1β.

3. The method as claimed in claim 1, wherein the T-cell mediated cytokines are IL-2, IFN-γ and IL-4.

4. A method of inhibiting T-cell surface markers, said method comprising step of contacting the T-cell or administering to a subject in need thereof an effective amount of colocynthin and/or its derivatives.

5. The method as claimed in claim 4, wherein the T-cell surface markers are CD4 and CD8.

6. A method of regulating immune response, said method comprising step of administering to a subject in need thereof a therapeutically effective amount of colocynthin and/or its derivatives.
